(19) United States
(12) Patent Application Publication
(10) Pub. No.: US 2023/0193201 A1
(43) Pub. Date: Jun. 22, 2023

(54) METHODS FOR PRODUCING IMMUNE CELL CULTURES

(71) Applicant: LONZA WALKERSVILLE, INC., Walkersville, MD (US)

(72) Inventors: Senthil RAMASWAMY, Walkersville, MD (US); Himavanth GATLA, Walkersville, MD (US)

(21) Appl. No.: 18/063,982

(22) Filed: Dec. 9, 2022

Related U.S. Application Data
(60) Provisional application No. 63/291,130, filed on Dec. 17, 2021.

(51) Int. Cl. C12N 5/0783

(52) U.S. Cl. CPC C12N 5/0636; C12N 2506/45; C12N 2521/10

(57) ABSTRACT

The present disclosure provides a method of producing an immune cell culture utilizing a fully closed system.

### FIELD OF THE INVENTION

The present disclosure provides methods for producing an immune cell culture in a fully closed system. In particular, the present disclosure relates to a process for upstream production and downstream processing of an immune cell culture.

### BACKGROUND OF THE INVENTION

Immune cell therapy is a class of disease treatment using genetically engineered immune cells to efficiently target and destroy cancerous cells. For example, adoptive cell therapy uses CAR T-cells expressing a chimeric antigen receptor designed to bind to certain proteins on cancer cells. Use of CAR T-cells in the treatment of cancer showed remarkable tumor specificity and robust anti-tumor immune responses, resulting in complete responses. To date, FDA has approved four autologous CAR T cells therapy products: tisagenlecleucel for acute lymphoblastic leukemia (Novartis, 2017), axicabtagene ciloleucel for large B cell lymphoma (Gilead, 2017), brexucabtagene autoleucel for mantle cell lymphoma in 2020 and for relapsed or refractory B-cell precursor acute lymphoblastic leukemia (ALL) in 2021 (Gilead), and lisocabtagene maraleucel for relapsed or refractory large B-cell lymphoma (Bristol Myers Squibb, 2021) (Young, C.M., C. Quinn, and M.R. Trusheim, Durable cell and gene therapy potential patient and financial impact: US projections of product approvals, patients treated, and product revenues. Drug Discov Today, 2021)

Although autologous CAR T cell therapies show remarkable efficacy, they suffer several limitations. Autologous CAR T cell therapy requires T cell harvest from the patients, followed by genetic modification to express CAR and expansion, which could take at least 2 weeks. During this process, progression of aggressive tumors could be lethal because the expansion of T cells depends on the quality attributes of the input and the inability to optimize the quality of patient’s T cells could lead to poor yield. Expansion of tumor infiltrating leukocytes provides an attractive strategy as the lymphocytes are primed against multiple tumor associated antigens. However, their expansion ex-vivo is not effective because of their exhausted phenotype and limited replicative potential. In addition to the above, prohibitive cost of the procedure presents a critical challenge for autologous cell therapies.

In contrast, allogeneic cell therapy could be available as an ‘off-the-shelf’ product to address the challenges of autologous cell therapy. Since matching of HLA-A, -B and -DR could potentially negate graft vs host disease (GVHD), cell therapy products generated from selected individuals could be applicable for wider population. Hence, cell banks could be generated from optimal T cell subpopulations of healthy individuals, decreasing the production cost, while increasing the applicability and effectiveness. Therefore, allogeneic cell therapy has the potential to break the limitations of autologous cellular therapy. Owing to the promise of allogeneic cellular therapy, clinical trials evaluating the efficacy of allogeneic, ‘off-the-shelf’, CAR T cells, targeting various tumor associated antigens, are in progress. Given the progress in cellular therapy, especially in the allogeneic front, requirement for industrial scale production of T cell products is inevitable.

While the number of CAR T cells per dose varies, estimates suggest the requirement of 3 trillion CAR T cells per year to treat hematological malignancies and 150 trillion CAR T cells for solid tumor malignancies. To meet the required batch sizes, scaling-up a static, 2D, flask-based cultures will result in added labor, lab footprint, variability, risk of contamination and poor process control. Because scale-up transfer kinetics are well characterized for stirred tank bioreactors (STRs), they provide an excellent option, meeting the demand. Furthermore, as STRs are a closed, automated, GMP compatible system with an in-process control, T cell manufacturing in STRs significantly decreases the labor, batch to batch variation and contamination risk. T cells are non-adherent, inherently cultured in-suspension as single cells. This makes suspension-based bioreactors suitable for their expansion, without the artificial need to adapt them to suspension culture condition and culture them in aggregates or attached to carriers. Similar to other cell types, expansion of T cells is sensitive to the buildup of cell metabolites, such as ammonia and lactate, in the culture media and hence requires media replenishment. Even though fresh nutrients are supplied and metabolites are removed using fed-batch cultures, inhibitory agents build up in the intervals between fed-batch media changes, could inhibit the growth, warranting continuous media change, through perfusion. The attributes that makes T cell suitable for expansion in 3D-based, suspension cultures also present a challenge for automated, continuous, media change. Because the diameter of T cells ranges from 5-10 mm, perfusion of T cell cultures has been difficult, with major responsible factors being frequent filter fouling and cell escape. Hence scale-up technology equipped with continuous media perfusion enabling T cell expansion is urgently needed.

### SUMMARY OF THE INVENTION

In some embodiments, a method of producing an immune cell culture in a fully closed system, comprising obtaining an immune cell, introducing the immune cell into a stirred-tank bioreactor comprising an immune cell complete medium, activating the immune cell with an activation reagent to produce an activated immune cell in the stirred-tank bioreactor, expanding the activated immune cell to produce an expanded immune cell culture in the stirred-tank bioreactor, exchanging a defined amount of fresh medium for spent medium via an alternating tangential flow filtration (ATF) connected to the bioreactor, depleting the expanded immune cell culture to produce a depleted immune cell culture in the stirred-tank bioreactor, harvesting the depleted immune cell culture to produce a harvested immune cell culture in the fully closed system and concentrating the harvested immune cell culture in the fully closed system, wherein the method results in less than 1% loss of the immune cell culture.

### BRIEF DESCRIPTION OF THE DRAWINGS

FIG. 1 shows a flow diagram for the production of an immune cell culture in accordance with embodiments hereof.

FIG. 2A shows the expansion of T-cells (Viable Cell Density (VCD)) in agitation at a constant 75 RPM, 50 RPM and increased to 100 RPM compared to 2D static culture as described in embodiments herein. FIG. 2B shows cell-fold expansion observed when agitation is initiated at 50 RPM and increased to 100 RPM. FIG. 2C shows T-cell expansion yield at agitation at 88 RPM and 65 RPM.

FIGS. 3A-3E show the expansion of activated T-cells in a stirred-tank bioreactor and continuous cell culture media perfusion as described in embodiments herein.

FIGS. 4A-4C show phenotypic characteristics of T-cells activated in stirred tank bioreactor with ATF mediated continuous perfusion as described in embodiments herein.

FIGS. 5A-5D show the functional status of T-cells activated in stirred tank bioreactor with ATF mediated continuous perfusion as described herein.

FIGS. 6A-6B show the efficiency of T-cell depletion in closed systems in accordance with embodiments hereof.

### DETAILED DESCRIPTION OF THE INVENTION

Allogeneic T cells are key immune therapeutic cells to fight cancer and other clinical indications. High T cell dose per patient and increasing patient numbers result in a clinical demand for large number of allogeneic T cells. This necessitates a manufacturing platform that can be scaled-up, while retaining cell quality. Allogeneic CAR T cells can be used as ‘off-the-shelf’ cell therapy and hold the promise to increase the applicability of the cell therapy product to population at a wider scale. Current estimates suggest the need for batch sizes of 2000 L to meet the demand of CAR T cells used for hematological and solid tumor malignancies. Given the large foot print, risk of contamination, variability and poor process control in static 2D cultures, stirred tank bioreactors provide an excellent platform for cell expansion of T cells, offering well characterized scale-up kinetics, in-process control and low risk of contamination. Translating 2D expansion processes into 3D expansion, in stirred tank bioreactors, was successfully shown for adherent cell types. However, absence of inherent perfusion capability of STRs and the small size of T cells (5-10 mM in diameter) proved to be formidable obstacles to achieve high yields of T cells in STRs.

Presented in this disclosure is a closed and scalable platform for T cell manufacturing to meet clinical demand. Upstream manufacturing steps of T cell activation and expansion are done in-vessel, in a stirred-tank bioreactor. T cell selection, which is necessary for CAR-T based therapy, is done in the bioreactor itself, thus maintaining optimal culture conditions through the se-lection step. Platform’s attributes of automation and performing the steps of T cell activation, expansion and selection in-vessel, greatly contribute to enhancing process control, cell quality, and to reduction of manual labor and contamination risk. In addition, the vi-ability of integrating a closed, automated, downstream process of cell concentration, is demonstrated. The presented T cell manufacturing platform has scale up capabilities, while preserving key factors of cell quality and process control. The present disclosure provides a GMP compatible, closed and scalable platform for T cell expansion in perfusion enabled STR. In addition, the present disclosure provides in-unit and potentially scalable cell depletion magnetic technology, avoiding a unit operation, lowering risk of contamination and labor.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or openended and do not exclude additional, unrecited elements or method steps. It is contemplated that any embodiment discussed in this specification can be implemented with respect to any method, device, system, and/or composition of the invention.

Technical and scientific terms used herein have the meaning commonly understood by one of skill in the art to which the present application pertains, unless otherwise defined. Reference is made herein to various methodologies and materials known to those of skill in the art.

In embodiments, provided herein is a method for producing an immune cell culture in a fully closed system. Suitably, the method is for automated upstream production and downstream processing of an immune cell culture.

In embodiments, provided herein is a method for introducing the immune cell into a stirred-tank bioreactor comprising an immune cell complete medium.

As referred to herein, the word “introducing” can mean adding the immune cell to a stirred-tank bioreactor or can indicate the presence of the immune cell within the stirred-tank bioreactor prior to beginning the method.

An “immune cell,” as produced and processed by the method upstream production and downstream processing, respectively, refers to cells of the immune system that are modified or primed (e.g., through co-culture with antigen presenting cells), resulting in cells that have a desired phenotype useful in treating, preventing or ameliorating one or more diseases in an animal, including a human. As used herein an “immune cell culture” refers to a collection of cells prepared by a method described herein, and can include a cell population for use in research or clinical trials, as well as for administration to a mammal, including a human patient, for a medical therapy. The genetically modified immune cell cultures that can be produced using the methods described herein can include mast cells, dendritic cells, naturally killer cells (NK-cells), B cell, T cells, etc.

In exemplary embodiments, the method comprises activating an immune cell with an activation reagent to produce an activated immune cell, expanding the immune cell, exchanging a defined amount of fresh medium for spent medium via an alternating tangential flow filtration (ATF) connected to the bioreactor, depleting the expanded immune cell culture to produce a depleted immune cell culture in the stirred-tank bioreactor, harvesting the depleted immune cell culture to produce a harvested immune cell culture in the fully closed system, and concentrating the harvested immune cell culture of in the fully closed system, wherein the method results in less than 1% loss of the immune cell culture.

In embodiments, the resulting loss of immune cell cultures is less than 0.99%, less than 0.95%, 0.9%, less than 0.85%, less than 0.80%, less than 0.75%, less than 0.70%, less than 0.65%, less than 0.60%, less than 0.55%, less than 0.50%, less than 0.45%, less than 0.40%, less than 0.35%, less than 0.30%, less than 0.25%, less than 0.20%, less than 0.15%, and less than 0.10%.

In embodiments, the immune cell is isolated from a population of peripheral blood mononuclear cells (PBMCs) immediately prior to obtaining the immune cell or is isolated from a population of PBMCs, stored for an extended period of time, and then thawed prior to obtaining the immune cell.

As referred to herein, to “isolate” an immune cell means to separate the immune cell from the matrix (cells, tissue, fluids, etc.) in which the product is produced. Suitably, isolation of an immune cell can comprise subjecting the immune cell to a series of mechanisms including but not limited to washes, magnetic applications, columns, filters, membranes, centrifuges and other isolation processes known in the art. The word “isolate” is synonymous with the word “purify” in this application.

In embodiments, the immune cell is derived from a population of pluripotent stem cells. In further embodiments, the population of pluripotent stem cells is a population of induced pluripotent stem cells (iPSCs), embryonic stem cells (ESCs), or combinations thereof.

As referred to herein, to “derive” an immune cell from a population of pluripotent stem cells means to generate the immune cell from in vitro from hematopoietic precursor cells present in hematopoietic zones within the bone marrow.

In embodiments, between 0.25 ×10⁶ T-cells/mL and 2 × 10⁶ T-cells/mL are introduced into the bioreactor. In other embodiments, between 0.1 ×10⁶ T-cells/mL and 0.5 × 10⁶ T-cells/mL are introduced into the bioreactor. In other embodiments, between 0.2 ×10⁶ T-cells/mL and 0.5 × 10⁶ T-cells/mL are introduced into the bioreactor. In other embodiments, between 0.3 ×10⁶ T-cells/mL and 0.6 × 10⁶ T-cells/mL are introduced into the bioreactor. In other embodiments, between 0.4 ×10⁶ T-cells/mL and 0.7 × 10⁶ T-cells/mL are introduced into the bioreactor. In other embodiments, between 0.5 ×10⁶ T-cells/mL and 0.8 × 10⁶ T-cells/mL are introduced into the bioreactor. In other embodiments, between 0.6 ×10⁶ T-cells/mL and 0.9 × 10⁶ T-cells/mL are introduced into the bioreactor. In other embodiments, between 0.7 ×10⁶ T-cells/mL and 1.0 × 10⁶ T-cells/mL are introduced into the bioreactor. In other embodiments, between 1.0 ×10⁶ T-cells/mL and 1.5 × 10⁶ T-cells/mL are introduced into the bioreactor. In other embodiments, between 1.5 ×10⁶ T-cells/mL and 1.7 × 10⁶ T-cells/mL are introduced into the bioreactor. In other embodiments, between 1.7 ×10⁶ T-cells/mL and 2.0 × 10⁶ T-cells/mL are introduced into the bioreactor.

In embodiments, the method produces an immune cell culture comprising about 10 × 10⁶ cells/mL to about 90 × 10⁶ cells/mL viable immune cells. In embodiments, the method produces an immune cell culture comprising about 90 × 10⁶ cells/mL to about 100 × 10⁶ cells/mL viable immune cells. In embodiments, the method produces an immune cell culture comprising about 100 × 10⁶ cells/mL to about 200 × 10⁶ cells/mL viable immune cells. In embodiments, the method produces an immune cell culture comprising about 200 × 10⁶ cells/mL to about 300 × 10⁶ cells/mL viable immune cells. In embodiments, the method produces an immune cell culture comprising about 300 × 10⁶ cells/mL to about 400 × 10⁶ cells/mL viable immune cells. In embodiments, the method produces an immune cell culture comprising about 500 × 10⁶ cells/mL to about 600 × 10⁶ cells/mL viable immune cells. In embodiments, the method produces an immune cell culture comprising about 600 × 10⁶ cells/mL to about 700 × 10⁶ cells/mL viable immune cells. In embodiments, the method produces an immune cell culture comprising about 800 × 10⁶ cells/mL to about 900 × 10⁶ cells/mL viable immune cells. In embodiments, the method produces an immune cell culture comprising about 1.0 × 10⁷ cells/mL to about 2.0 × 10⁷ cells/mL viable immune cells.

In embodiments, the immune cell is activated with an activation reagent to produce an activated immune cell in the stirred-tank bioreactor. In further embodiments, activating the immune cell comprises stirring the medium with the activation reagent for a period of about 72 hours at 37° C. In embodiments, the immune cell culture is stirred at a tip speed of 0.15 to 0.5 revolutions per minute (RPM). In embodiments, the immune cell culture is stirred at a tip speed of 0.6 to 0.8 revolutions per minute (RPM). In embodiments, the immune cell culture is stirred at a tip speed of 0.8 to 1.0 revolutions per minute (RPM).

In embodiments, the immune cell culture medium has a pH between about pH 5.0 and about pH 7.5 during the activation period. In embodiments, the immune cell culture medium has a pH between about pH 5.0 and about pH 5.5 during the activation period. In embodiments, the immune cell culture medium has a pH between about pH 5.5 and about pH 6.0 during the activation period. In embodiments, the immune cell culture medium has a pH between about pH 6.0 and about pH 6.5 during the activation period. In embodiments, the immune cell culture medium has a pH between about pH 6.5 and about pH 7.0 during the activation period. In embodiments, the immune cell culture medium has a pH between about pH 7.0 and about pH 7.5 during the activation period.

Suitably, the activation reagent comprises soluble antibody complexes. In embodiments, the activation reagent comprises an antibody that is a soluble antibody, including at least one of an anti-CD3 antibody and an anti-CD28 antibody. Exemplary antibodies include OKT3.

In other embodiments, the activation reagent comprises an antibody or a dendritic cell. In embodiments, the antibody is immobilized on a surface, which can include a polystyrene plastic, silicone or other surface, including for example, the surface of a bead.

In embodiments, the activated immune cell is expanded to produce an expanded immune cell culture in the stirred-tank bioreactor. As described herein, the methods of expanding the cells suitably include at least one or more of adding fresh medium to the bioreactor, feeding, washing, monitoring and adjusting the conditions of the immune cell culture. In further embodiments, the expanding further comprises sampling the expanding immune cell culture, determining a cell growth and fold expansion of the expanding T cell culture and exchanging a defined amount of fresh medium for spent medium based on the cell growth and fold expansion. Exemplary conditions include temperature, a pH level, a glucose level, an oxygen level, a carbon dioxide level, and an optical density.

The various methods described herein are conducted in a manner such that the oxygen level of the expanded immune cell culture is optimized for the immune cell culture. This optimization allows for production of a large number of viable cells having the desired phenotypic characteristics, including, as described herein, the promoting of a desired cell phenotype. In embodiments, oxygen level or concentration is optimized by the alternating tangential flow filtration system recirculating T cell complete media through an oxygenation component during one or more of steps (d) to (f).

In further embodiments, the alternating tangential flow filtration system recirculates nutrients, waste, released cytokines, and/or dissolved gasses during the various method processes. This recirculation helps aid in the production of a large number of viable cells having the desired phenotype(s).

Other mechanisms for optimizing the expansion conditions for the cells include modifying and controlling the flow rate of the media provided to the cells. As the cells begin to grow, the circulation rate of the media provided is increased, which improves gas exchange and allows oxygen and carbon dioxide to either enter or leave the cell culture, depending on the conditions of the cells and the requirements at the time.

In embodiments, the system is configured to perform several rounds of one or more of feeding, washing and monitoring, and in embodiments, selecting of the expanded immune cell culture. These various activities can be performed in any order, and can be performed alone or in combination with another activity. In embodiments, concentrating of the cells comprises centrifugation, supernatant removal following sedimentation, or filtration. Suitably, the optimization process further includes adjusting parameters of the centrifugation or filtration, suitably in a self-adjusting process. Depletion of the expanded cell culture can be carried out by, for example, magnetic separation, filtration, adherence to a bead, plastic or other substrate, etc.

In embodiments, the fresh medium is exchanged for spent medium at a rate of one vessel volume per day (VVD) when the viable cell density of the expanding immune cell culture is greater than 1.5×10⁶ cells/mL. In embodiments, the fresh medium is exchanged for spent medium at a rate of one vessel volume per day (VVD) when the viable cell density of the expanding immune cell culture is greater than 1.4 ×10⁶ cells/mL. In embodiments, the fresh medium is exchanged for spent medium at a rate of one vessel volume per day (VVD) when the viable cell density of the expanding immune cell culture is greater than 1.3 ×10⁶ cells/mL. In embodiments, the fresh medium is exchanged for spent medium at a rate of one vessel volume per day (VVD) when the viable cell density of the expanding immune cell culture is greater than 1.2 ×10⁶ cells/mL. In embodiments, the fresh medium is exchanged for spent medium at a rate of one vessel volume per day (VVD) when the viable cell density of the expanding immune cell culture is greater than 1.0 × 10⁶ cells/mL.

In embodiments, depleting the immune cell culture comprises adding surface-activated magnetic beads to the immune cell culture after the expanding, stirring the expanded immune cell culture and beads for about 30 minutes, isolating a population of target cells from the expanded immune cell culture with a magnet. In other embodiments, depleting the immune cell culture comprises physical separation methods known in the art including using counterflow centrifugal elutriation, fractionation on density gradients, or the differential agglutination with lectins followed by resetting with sheep red blood cells. In other embodiments, depleting the immune cell culture comprises immunological methods known in the art utilizing antibodies, either alone, in conjunction with homologous, heterologous, or rabbit complement factors which are directed against the T cells. In other embodiments, depleting the immune cell culture comprises using a combination of physical separation methods and immunological methods described herein.

In exemplary embodiments, the stirred tank bioreactor contains the immune cell culture medium prior to starting the method. In other embodiments, fresh immune cell culture medium can be added separately following the start of the method of production, or at any suitable time during the process.

In other embodiments, provided herein is method for promoting a preferred phenotype of an immune cell culture, the method comprising activating an immune cell culture with an activation reagent to produce an activated immune cell culture in a stirred tank bioreactor and expanding the activated immune cell to produce an expanded immune cell culture in the stirred tank bioreactor, wherein the activating and expansion conditions promote the phenotype and functional status of the immune cell culture. Exemplary phenotypes include stemness, ability to produce cytokines, central memory, effector memory, and naïve/stem memory. Exemplary functional statuses include cytokine production As described herein, the methods are suitably performed by a fully enclosed, automated cell engineering system.

In embodiments, the activating conditions provide a substantially undisturbed immune cell culture allowing for stable contact between the activation reagent and the immune cell culture. As described herein, it has been found that allowing the cells to activate under substantially undisturbed conditions via the use of a stirred tank bioreactor provides an environment where the cells can be homogenously contacted with the activation reagent, as well as interact with the necessary nutrients, dissolved gasses, etc., to achieve the desired and promoted phenotype.

The methods described herein can influence the characteristics of the final immune cell culture product by selecting an appropriate activation method to provide the preferred phenotype. For example, activation utilizing a bead-based process as described herein promotes a more balanced CD4:CD8 ratio, whereas use of a soluble anti-CD3 promotes a higher population of CD8 than CD4. Other levels of CD8 and CD4 can also be provided using the methods described herein. In exemplary embodiments, as described herein, the methods can be utilized to prepare CAR T cells. Suitably, the methods can be utilized to promote a phenotype of the CAR T cells that has a ratio of CD8+ cells to CD4+ of about 0.1:1 to about 10:1, including a ratio of CD8+ cells to CD4+ cells of about 0.5:1 to about 5:1, about 0.8: to about 3:1, or about 1:1, about 2:1, etc.

As described herein, it has been surprisingly found that allowing the cells to expand under conditions where they are not shaken (i.e., not rotated or shaken in order to cause the cells to flow over top of one another), the methods provide optimal cell characteristics, including high viable cell yield and desired phenotypes. It has been determined that a large, un-shaken cell culture chamber, can provide homogenous access of the cells to the necessary reagents, nutrients, gas exchange, etc., while removing cellular waste, without the requirement to shake or disturb the cells to achieve the desired outcome. In fact, as described herein, it has been found that such methods for the automated production of genetically modified immune cells produce higher numbers of viable cells, greater numbers/ratios of desired cells types, and more robust cellular characteristics, as compared to methods that utilize cellular shaking, for example, as described in Miltenyi et al., “Sample Processing System and Methods,” U.S. Pat. No. 8,727,132.

In embodiments, the various steps of the method are performed by a fully closed system and are optimized via a process to produce the immune cell culture.

Suitably, the method described herein comprises one or more sensors and/or mechanisms to detect and/or adjust one or more of the following: temperature, a pH level, a glucose level, an oxygen level, a carbon dioxide level, and an optical density of the immune cell culture.

As used herein, “bioreactor” can include a fermenter or fermentation unit, or any other reaction vessel. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. The methods described herein can be utilized in connection with any suitable bioreactor including but not limited to stirred tank, airlift, fiber, microfiber, hollow fiber, ceramic matrix, fluidized bed, fixed bed, and/or spouted bed bioreactors. Any suitable reactor diameter can be used. In some embodiments, the bioreactor allows for stirring or mixing of the liquid medium by continuous and repeated movement within.

In embodiments, the bioreactor can have a volume capacity of about 1 L to about 2000 L. Non-limiting examples include a volume capacity of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316 L or any other suitable stainless steel) and Inconel, plastics, and/or glass.

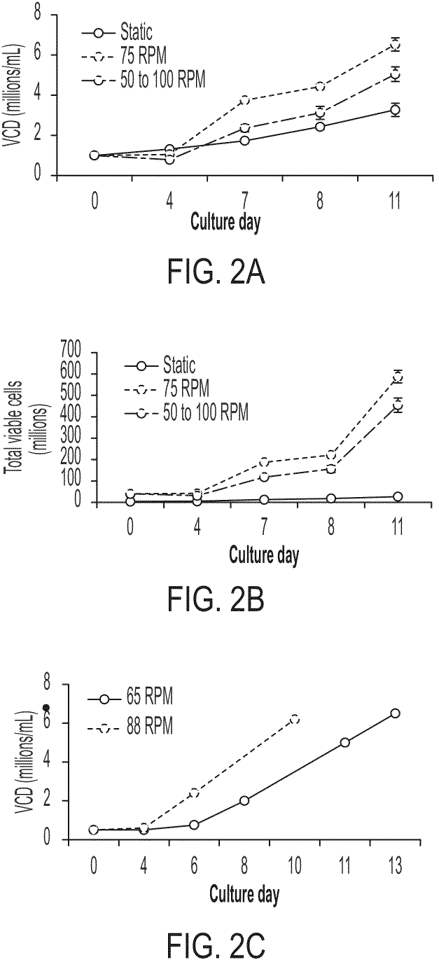

Stirred tank bioreactors utilize agitation as a mechanism to ensure even distribution of gasses and nutrients. To determine whether T cells could be expanded in agitation, the activated and cultured T cells at different agitation rates in spinner flasks were compared to the cells expanded in 2D static flask. Cell counts, performed at various time points throughout the run, indicated that the Viable Cell Density (VCD) and total number of T cells in 2D static culture is consistently lower than agitated conditions (FIGS. 2A and 2B). Agitation at a constant 75 RPM resulted in higher VCD of T cells, compared to the VCD when agitation is initiated at 50 RPM and increased to 100 RPM on day 5 (FIG. 2A). Furthermore, agitation at 75 RPM resulted in a 15-fold T cell expansion, compared to 10-fold cell expansion observed when agitation is initiated at 50 RPM and increased to 100 RPM (FIG. 2B). Maintaining the fed batch media change regime and the range of the tip speed tested in spinner flask, the expansion of T cells in 1 L stirred tank bioreactor (STR) was evaluated at two different agitation rates. As shown in FIG. 2C, agitation at 88 RPM resulted in higher viable cell density of T cells, compared to agitation at 65 RPM.

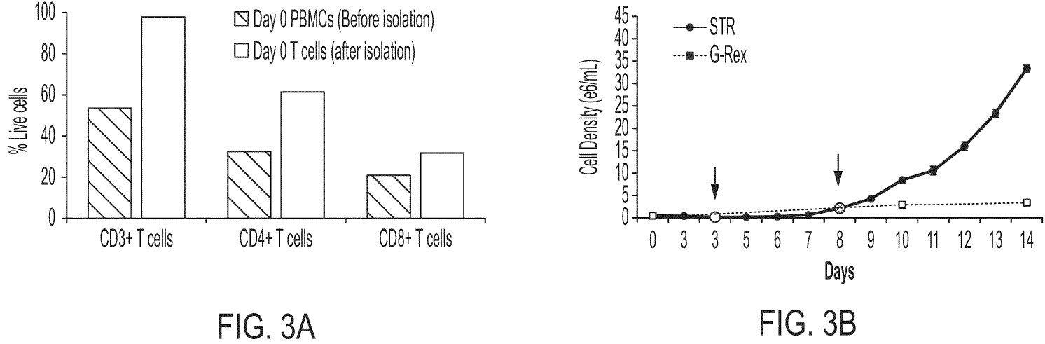

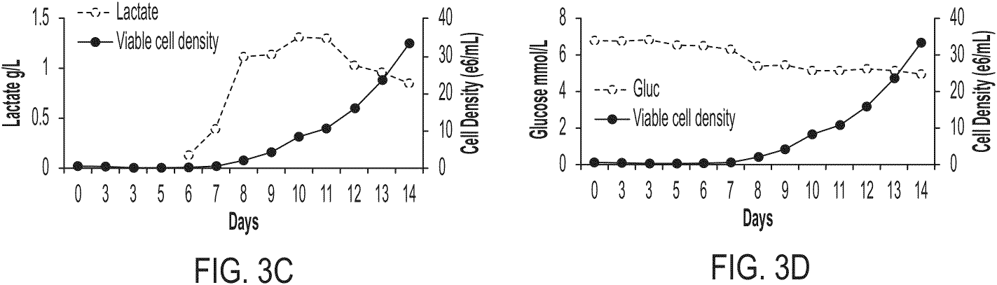

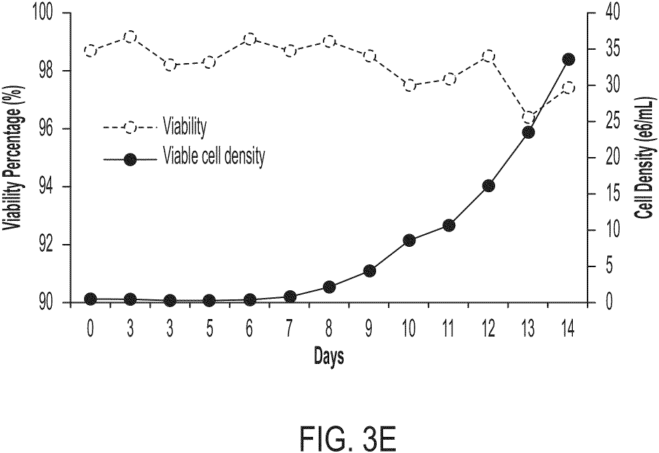

Increase in cell density is accompanied by nutrients depletion and inhibitory metabolites accumulation. Continuous media perfusion provides optimal culture conditions and enables cell expansion. To evaluate if continuous media perfusion can be performed using alternating tangential flow (ATF) without cell loss, the effect of 24 hour media perfusion by ATF on T cells, inoculated at 3.0 × 10⁶ cells/mL in 2 L culture media in a 3 L STR was tested. As shown in Table 1, no notable decrease in the VCD of the T cells, 24 hours after perfusion was observed. This was accompanied by the absence of T cells in the waste bag, indicating that ATF-mediated media change did not result in cell loss. Furthermore, evaluation of the weight of the waste bag 24 hours after perfusion showed that a continuous media perfusion of 1 vessel volume per day (1 VVD) was achieved without filter fouling. To evaluate if T cell expansion can be achieved in STR with continuous media perfusion, CD3+ T cells were isolated from peripheral blood mononuclear cells (PBMNCs), inoculated into the stirred tank bioreactor and were activated as described in Materials and Methods. Phenotypic evaluation of the cells after isolation showed that 98% of the cells are CD3+ (FIG. 3A) with a viability of >98% (data not shown). Post inoculation and activation in STR, expansion of T cells over 14 days was monitored, showing T cell VCD reaching 2.0 × 10⁶ cells/mL on day 8 (FIG. 3B). With the increase in cell density, a concurrent increase in the lactate levels was observed (FIG. 3C). Cell retention and media exchange using ATF at 1 VVD curtailed lactate buildup and enabled the viable cell density to reach 33.5 × 10⁶ cells/mL on day 14 (FIG. 3C). In comparison, T cells isolated from the same donor and cultured in static mode, in G-Rex®, resulted in VCD of 3.4 × 10⁶ cells/mL on day 14 (FIG. 3B). In addition to avoiding the lactate accumulation, ATF mediated perfusion at 1 VVD enabled nutrient replenishment, as shown by stable glucose levels during cell expansion in FIG. 3D. Furthermore, agitation in the stirred tank bioreactor in combination with ATF mediated cell movement did not lead to cell death, demonstrated via stable >96% cell viability (FIG. 3E).

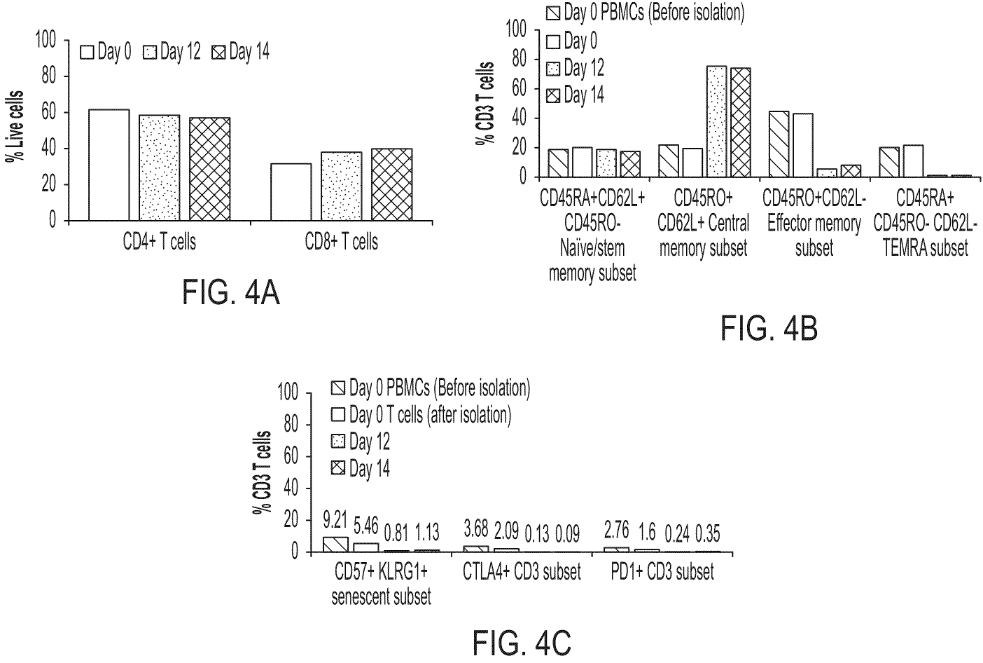

Achieving high cell expansion folds is a key for process scale up. This attribute, however, does not hold the promise to achieve performance if cell quality is not optimal. Assesment of CD4:CD8 T cell ratio for T cells expanded in the stirred tank bioreactor showed that the CD4:CD8 T cells ratio at inoculation was maintained during expansion with a gradual shift towards CD8+ T cells over time (FIG. 4A). To determine the impact of expansion in stirred tank bioreactor on the phenotype of the T cells, the T cell phenotype during and after the expansion, in comparison to the T cell phenotype before inoculation was evaluated. As shown in FIG. 4B, T cell expansion resulted in ~80% central memory T cells, <10% effector memory subset and ~15% naïve/stem memory subset. Furthermore, the expansion did not result in accumulation of terminally differentiated (FIG. 4B), senescent or exhausted T cells (FIG. 4C).

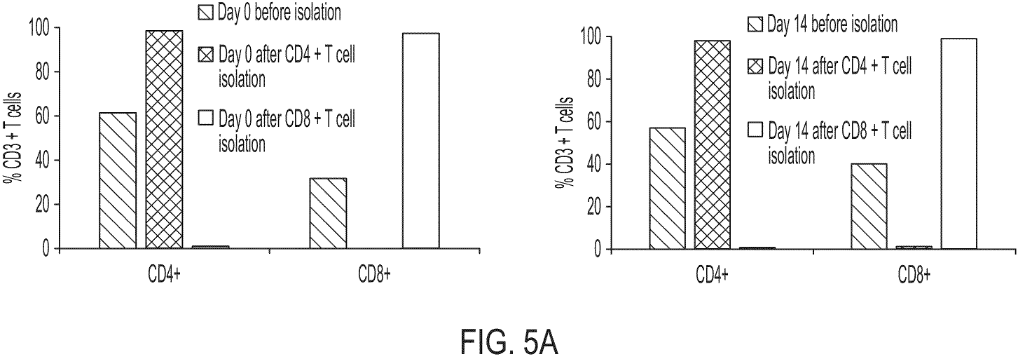

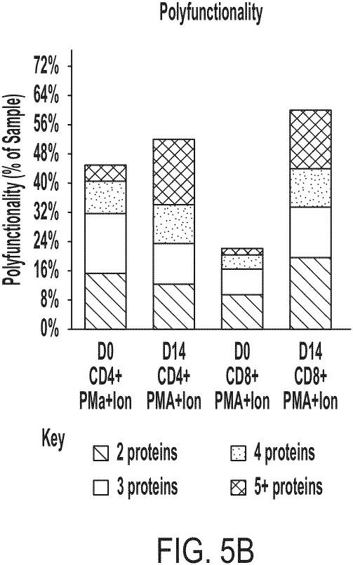

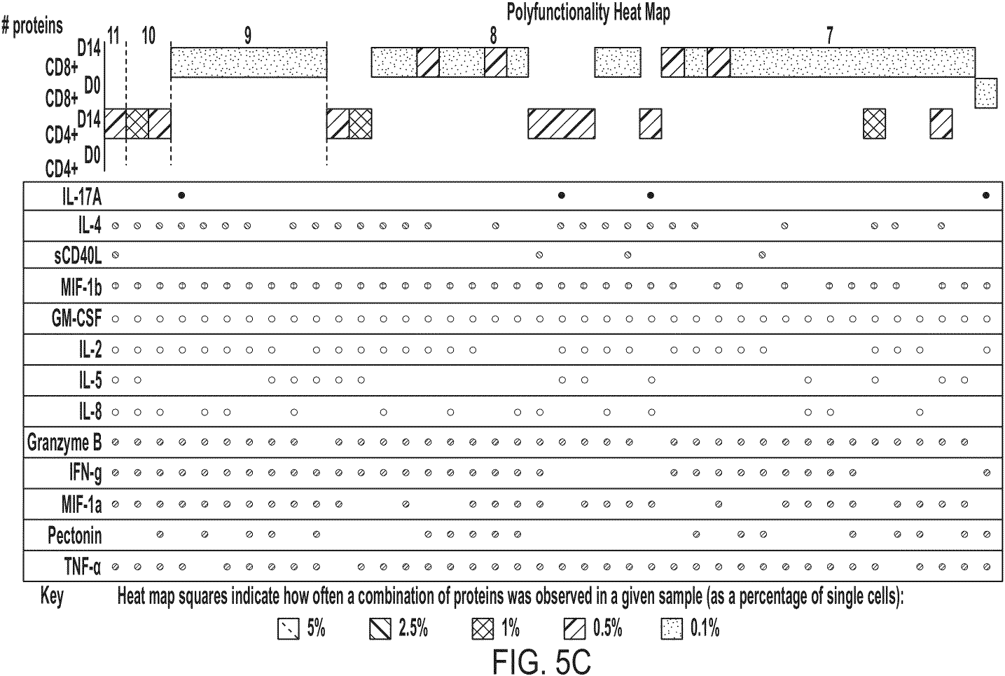

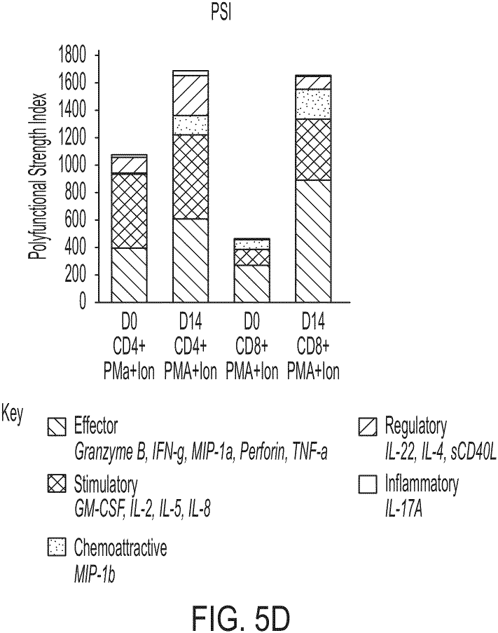

To assess the functional status of T cells after expansion in a stirred-tank bioreactor, the ability of the T cells to produce cytokines after stimulation was evaluated. Isolation of CD4 + and CD8 + T cells from T cell samples obtained on day 0 and day 14 resulted in >98% pure populations (FIG. 5A). The cells were stimulated, stained and evaluated for cytokine production as described in Materials and Methods. As shown in FIG. 5B, the number of cells producing multiple cytokines — indicating polyfunctionality — is maintained throughout the expansion. In comparison to day 0, day 14 sample showed increased number of cells producing more than 5 cytokines. Even though at low frequency, day 14 T cells produced the highest number of cytokines, with CD4 + T cells producing 11 cytokines and CD8 + T cells producing 9 cytokines (FIG. 5C). Moreover, cytokine signature-based categorization of the cell types suggests an increase in effector polyfunctional strength index whilst retaining stimulatory signature (FIG. 5D).

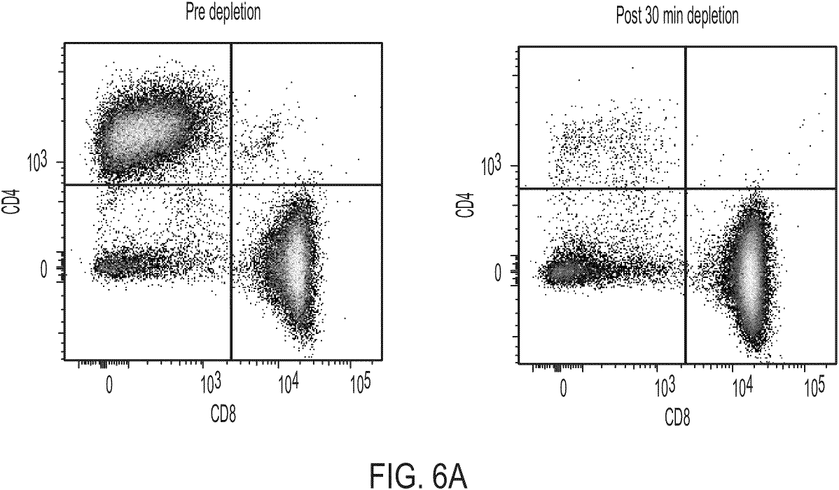

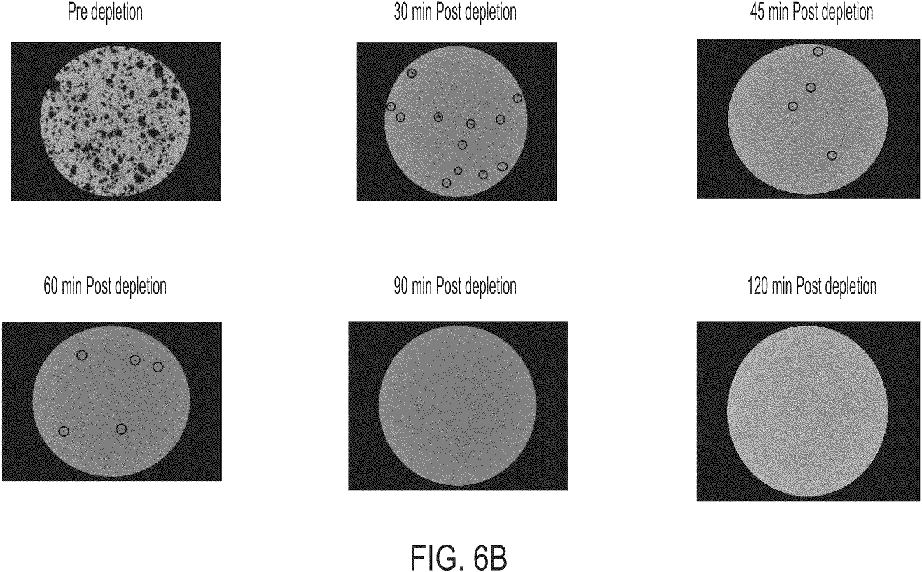

As mentioned above, depletion of TCR positive cells is needed to enable allogeneic CAR T cell-based therapy. The concentration of undesired T cells varies based on the cell editing technology and delivery platform, and might result in low or high concentrations after expansion. As a proof of concept, the depletion of CD4+ T cells at low (17%) and high concentrations (52%) was evaluated. 30 mins of CD4+ T cell depletion using proprietary magnetic technology resulted in >99% depletion in lower cell concentrations and ~97% in higher concentrations (Table 2 and FIG. 6A). To assess the time required to deplete CD4 + T cells and beads at higher density, a time course of bead depletion was performed. As shown in FIG. 5B, application of magnetic field for 90 mins depletes the beads, as shown by visual bead count, post sampling. Evaluation of residual bead percentage showed to result in the presence of <0.001% beads after 120 mins of bead depletion with magnets without a loss in cell viability and CD8+ T cells (Table 3 and FIG. 6B).

Two mutually exclusive systems - ekko™ (Millipore-Sigma) and kSep400 (Sartorius) were evaluated for cell concentration post closed harvest. Closed cell harvest from the STR into harvest bag was done as described in Materials and Methods. Cells from the bag were transferred in a closed manner to each of the concentration equipment. As shown in Table 4, cell concentration using ekkoTM resulted in concentration of 7 fold, 0% loss in cell viability with a final cell viability of 98.5% and cell recovery of 79%. Similarly, cell concentration using kSep 400 resulted in concentration of 7.65 fold, 6% loss in cell viability with a final cell viability of 89.4% and cell recovery of 69%.

### EXAMPLES

### Isolation of T Cells From PBMCs

Human PBMCs (Lonza Cat #4W-270C) were thawed rapidly in a 37° C. water bath till a small bit of ice was left in the vial. Thawed cells were added dropwise to EasySep™ buffer (Stem Cell Technologies Cat #20144). The cells were centrifuged at 300 RCF for 5 minutes at Room Temperature (RT). The supernatant was discarded and the cells were reconstituted in 50 mL of EasySep™ buffer. Cell concentration and viability were evaluated-ed using the NucleoCounter NC-200 (Chemometec, Denmark). The cells were centrifuged again at 300 RCF for 5 minutes at RT. The supernatant was discarded and the cells were reconstituted at 50 × 106 cells/mL in X-VIVOTM 15 Serum-free Hematopoietic Cell Medium (Lonza Cat #04-418Q). A sample was aliquoted for immune-phenotype staining. T cells were isolated using T cell isolation kit (Stem Cell Technologies Cat #17951) by following manufacturer’s protocol. Post T cell isolation, cell concentration and viability were evaluated using the NucleoCounter NC-200 (Chemometec, Denmark), and a sample was aliquoted for immune-phenotype staining.

### T Cell Activation and Expansion in Spinner Flasks

T cell complete media was prepared by adding Human AB serum (Sigma Cat #H4522) to a final concentration of 5% and recombinant human IL-2 (Peprotech Cat #200-02) to a final concentration of 50 ng/mL to X-VIVOTM 15 (Lonza Cat #04-418Q). After T cell isolation (as described in 4.1), cells were seeded in spinner flasks at 1 × 106 cells/mL in 40 mL of T cell complete media. Cells were stimulated with ImmunoCultTM Human CD3/CD28 T-cell activator (Stem Cell Technologies Cat #10991) for 3 days following the manufacturer’s instructions. Media was added on day 5 to adjust the cell density to 1 × 106 cells/mL and media was doubled on day 8. Agitation in one condition of the spinner flasks was maintained at 75 RPM for the entirety of 11 days and agitation in the other was started at 50 RPM through days 1-4 followed by 100 RPM through days 5-11. Cell count and viability were determined by sampling on culture day 4, 7, 8 and 11.

### T Cell Activation and Expansion in G-Rex®

T cell complete media was prepared by adding Human AB serum (Sigma Cat #H4522) to a final concentration of 5% and recombinant human IL-2 (Peprotech Cat #200-02) to a final concentration of 50 ng/mL to X-VIVOTM 15 (Lonza Cat #04-418Q). After T cell isolation (as described in 4.1), a 1L G-Rex® (Wilson Wolf Cat #G-Rex®100M-CS) was inoculated with T cells at 0.5 × 106 cells/mL in 300 mL of T cell complete media. T cell activation was performed in the G-Rex® using ImmunoCultTM Human CD3/CD28 T-cell activator (Stem Cell Technologies Cat #10991) for 3 days following the manufacturer’s instructions. T cell culture in the G-Rex® was maintained at 37oC, 5% CO2 humidified atmosphere. After T cells were activated for 3 days, 700 mL of T cell complete media was added and was cultured till day 14.

### T Cell Activation and Expansion in a Stirred Tank Bioreactor

The BioBlu single-use bioreactor vessel was set up according to manufacturer’s instructions (Eppendorf, 1386000300). Briefly, the 1 L vessel was equipped with probes re-quired for online monitoring (Mettler Toledo) of key parameters including percentage of dissolved oxygen (DO), pH and temperature. The bioreactor was controlled using a G3 Lab Universal controller (Thermo Fisher Scientific). T cell complete media was prepared by adding Human AB serum (Sigma Cat #H4522) to a final concentration of 5% and recombinant human IL-2 (Peprotech Cat #200-02) to a final concentration of 50 ng/mL to X-VIVOTM 15 (Lonza Cat #04-418Q). Prior to inoculation, 400 mL of T cell complete media was introduced into the bioreactor and was equilibrated with air. After T cell isolation, the bioreactor was inoculated with 200 × 106 T cells at a seeding density of 0.5 × 106 cells/mL. T cell activation was performed in the bioreactor using ImmunoCultTM Human CD3/CD28 T-cell activator (Stem Cell Technologies Cat #10991) for 3 days following the manufacturer’s instructions. T cell culture in the bioreactor was maintained at 37oC, 88 RPM agitation (except otherwise mentioned) and pH < 7.2. After T cells were activated for 3 days, 600 mL of T cell complete media were added. To determine cell growth and fold expansion, 15 mL samples were taken in duplicates at various time points along the run and the Nucle-oCounter NC-200 (Chemometec, Denmark) was used to measure the cell number and viability. When the viable cell density of T cells has reached 2.0 × 106 cells/mL, perfusion with fresh T cell complete media was initiated at a rate of one Vessel Volume per Day (VVD). On the indicated days, samples were used for immune-phenotypic analysis, and single cell secretome analysis. To monitor the changes in key metabolites, 5 mL samples were taken from the bioreactor at various time points along the run. Offline monitoring to determine changes in parameters such as pH and key nutrients was performed using the BioProfile FLEX Analyzer (Nova Biomedical). As shown in Table 1, cell concentration, viability, cell presence in waste bag and volume in and out of bioreactor were evaluated

TABLE 1

| Variable | Before perfusion | 24h after perfusion |
|---|---|---|
| Cell concentration (cells/mL) | 3 × 10⁶ | 2.5 × 10⁶ |
| Cell viability (percentage) | 94 | 96.2 |
| Cell concentration in waste (cells/mL) | 0 | 0 |
| Volume in media bag (L) | 2 | 0 |
| Volume in waste bag (L) | 0 | 2 |

### Continuous Media Perfusion

Cell retention and continuous media perfusion was performed using XcellTM ATF 2 single-use device (Repligen Cat #suATF2-S02PES). ATF was aseptically connected to the bioreactor on Day 0 and the ATF column was wetted using X-VIVOTM 15 Serum-free Hematopoietic Cell Medium (Lonza Cat #04-418Q). On day 8, when the cells have reached 2.0 × 106 cells/mL, perfusion was enabled at 0.5 Liters Per Minute (LPM) ATF rate. Media in and media out were set to 0.7 mL/min, maintaining 1 VVD.

### CD4+ T Cell Depletion

After cell expansion, on day 14, perfusion was stopped and the required number of DynabeadsTM CD4 (Thermo Fisher Scientific Cat #11145D) were washed in isolation buffer (DPBS supplemented with 0.1% human AB serum and 2 mM EDTA) as per manufacturer’s instructions. The beads were added to the bioreactor and were incubated for 30 min in agitation. Incubation with magnets was performed for required time period. At indicated times, 10 mL of samples were drawn out of the bioreactor for taking pictures under 20x magnification of Rebel light microscope (Echo, USA), calculation of residual bead concentration (see 4.9), staining for immune-phenotypic analysis (see 4.8), evaluation of viable cell concentration and cell viability (NucleoCounter NC-200). As seen in table 2, efficiency of CD4+ T cell depletion for 30 minutes at low and high concentrations was evaluated.

TABLE 2

| Parameter | Run 1 Low % CD4+ T cells | | Run 2 High % of CD4+ T cells | |
|---|---|---|---|---|
| | Before CD4+ T cell depletion | After CD4+ T cell depletion | Before CD4+ T cell depletion | After CD4+ T cell depletion |
| CD4+ T cell % | 17% | 0% | 52.4% | 1.64% |
| Depletion | | >99% | | 96.87% |
| CD8+ T cell % | 80% | 97% | 39.8% | 85.4% |

Table 3 displays the efficiency of bead depletion. Time course of residual bead percentage, loss in cell viability and CD8+ T cells were evaluated.

TABLE 3

| Magnetic incubation (mins) | Residual bead percentage | Decrease in viability (percentage) | Loss in CD8+ T cells (percentage) |
|---|---|---|---|
| Pre depletion | 78.370 | 0 | |
| 30 | 1.021 | 3.9 | 0 |
| 45 | 0.107 | 3.1 | 0 |
| 60 | 0.034 | 2.4 | 0 |
| 90 | 0.009 | 2.3 | 0 |
| 120 | 0.001 | 4.9 | 0 |

### Harvest of T Cells From Stirred Tank Bioreactor

T cells in the 1 L bioreactor were collected on day 14 of total cell culture. With the connection between bioreactor and ATF closed and continuous agitation, the cell solution in the bioreactor was pumped into a sterile 1 L bag. The same 1 L bag was connected to the harvest line of the ATF and the cell solution present in the ATF was harvested. 15 mL of cell solution in the harvest bag was sampled for immune-phenotypic analysis (see 4.8), evaluation of viable cell concentration and cell viability (NucleoCounter NC-200).

### Downstream Processing

A bag containing the T cell suspension harvested from the bioreactor was sampled in triplicate, and the viabilities and cell densities were then deter-mined using a NucleoCounter NC-200. The average viable cell density (VCD) was used to calculate the concentrated volume that would be harvested by the kSep (Equation 1, see Appendix A). A kSep (Sartorius) was fitted with a 400.50 rotor, which functions as a 1/3.5 scale-down model for the kSep400. The associated 400.50 single-use kits (chamber set and valve set) were then installed. A solution of PlasmaLyte-A (Baxter) and (0.25%) human AB serum (Sigma Cat #H4522) was used to prime the system A static centrifugation speed of 1000 g was used. The fluidized bed was established at 24 mL/min flow rate for 60 min and was harvested at 120 mL/min into a harvest bag. For the entirety of the concentration process, 5 mL samples were drawn from the stream exiting the kSep chamber and tested using the NucleoCounter NC-200 (Chemometec, Denmark) to monitor the amount of cells escaping the fluidized bed. After 1 L of cell suspension was processed, the concentrated cells were harvested. The volume of the concentrate was verified, and samples were taken to determine viability and cell density. The remaining concentrate was cryopreserved.

For concentration by ekkoTM, an ekkoTM single use cartridge was installed and the chamber was primed with 100 mL of wash buffer (A solution of PlasmaLyte-A (Baxter) and (0.25%) human AB serum (Sigma Cat #H4522). The feed was recirculated to from acoustic fluid bed at 120 W and a flow rate of 70 mL/min. After 1 L of cell suspension was processed, the concentrated cells were harvested in 2 cycles. The volume of the concentrate was verified, and samples were taken to determine viability and cell density. The remaining concentrate was cryopreserved.

Table 4 shows the concentration of cells using closed systems. Fold concentration, cell viability and recovery % were evaluated.

TABLE 4

| Variable | ekko™ | kSep |
|---|---|---|
| Fold concentration | 7X | 7.65X |
| Cell viability | 98.5% | 89.4% |
| Loss in viability | 0% | 6% |
| Cell recovery | 79% | 69% |

### Cryopreservation

Human T cells were suspended in cryopreservation solution (CS10, Biolife Solutions Inc, 210102)). Cryovials were cryopreserved by Cryomed™ Controlled-rated Freezer (Thermo Fisher Scientific, Model 7456) and subsequently stored in liquid nitrogen until use.

### Flow Cytometry

Quantitative detection of T cell differentiation, senescence and exhaustion status was performed using flow cytometry. Briefly, 300,000 cells were live-stained for the cell surface markers: CD62L (Biolegend Cat #304806), CD45RA (Biolegend Cat #304108), CD45RO (Biolegend Cat #304218), CD3 (BD Biosciences Cat #564713), CD4 (CD Biosciences Cat #560158), CD8 (Biolegend Cat #301028), KLRG1 (Biolegend Cat #367716), CTLA4 (BD Biosciences Cat #563931), PD-1 (BD Biosciences Cat #564324), CD57 (Biolegend Cat #393304) and ZOMBIE 421 (Biolegend Cat #423114). The samples were processed using FACS CelestaTM (Becton Dickinson), and data was acquired using the BD FACS Diva Software followed by analysis using FlowJo v10 software (FlowJo).

### Residual Bead Percentage

From each sample, 1 ml was dispensed into each of 3 tubes. The tubes were centrifuged at 700 RCF for 5 min at room temperature. The cell pellet was resuspended in 1 mL of DPBS. To the cell solution, 4 mL of lysis buffer (1:1 ratio of DPBS and sodium hypo-chlorite) was added, mixed and left at room temperature for 5 min. The tubes were centrifuged at 700 RCF for 5 min at room temperature and 4.95 mL of supernatant was re-moved. The remaining 50 µL was mixed well and 10 µL was dispensed into each side of the hemocytometer. The number of beads in 4 corner squares were counted. The counting was repeated for the 2nd side of the hemocytometer. The remaining solution in the tube was mixed well and the counting was performed till the solution in the tube was completely counted. The residual bead count was calculated using Equation 2, see Appendix A. Residual bead percentage with respect to the viable cell density of the sample was calculated using Equation 3, see Appendix A.

### T Cell Polyfunctionality Analysis Using Isoplexis

During the bioreactor run, 15 ml of samples were drawn for multitude of analysis. At indicated time points, 1 mL of solution was centrifuged at 500 RCF for 5 min at room temperature. The supernatant was transferred into a new sterile 1.5 mL sterile tube and the pellet was cryopreserved as described in section 4.7. The cells were thawed as described in 4.1 and were recovered overnight at 5% CO₂ and 37° C. in T cell complete media supplemented with 10 ng/mL of human recombinant IL-2. CD4+ and CD8+ cell enrichment was performed as described in 4.13. The two distinctive T cell populations were stimulated for 5 hours in 50 ng/mL phorbol 12-myristate 13-acetate (PMA) (Sigma-Aldrich Cat #P8139) and 1 ug/mL Ionomycin (Sigma-Aldrich Cat #I0634). The cells were loaded into the single-cell secretome barcode chip (Isoplexis Cat #PANEL-1001-8) for single-cell secretomics evaluation. A single cell functional profile was determined for each T cell type. Profiles were categorized into effector (Granzyme B, IFN-y, MIP-1a, Perforin, TNF-α, TNF-β, stimulatory (GM-CSF, IL-2, IL-5, IL-7, IL-8, IL-9, IL-12, IL-15, IL-21), regulatory (IL-4, IL-10, IL-13, IL-22, TGF-β1, sCD137, sCD40L), chemo attractive (CCL-11, IP-10, MIP-1β, RANTES), and inflammatory (IL-1b, IL-6, IL-17A, IL-17F, MCP-1, MCP-4) groups.

### Isolation of CD4 + and CD8 + T Cells

CD4 + and CD8 + T cells were isolated from CD3 + T cells using CD4 + microbeads, human (Miltenyi Biotec Cat #130-045-101) and CD8 + microbeads, human (Miltenyi Biotec Cat #130-045-201). Briefly, the cells were labelled with specific microspheres and were passed through MACS LS columns (Miltenyi Biotec Cat #130-042-401) placed on the mi-diMACS Separator (Miltenyi Biotec Cat #130-042-302) and MACS Multistand (Miltenyi Biotec Cat #130-042-303). Enriched cells trapped in the column were plunged into a fresh collection tube, washed and were resuspended in T cell complete media. Samples were collected before and after isolation for flowcytometric analysis as described in 4.10.

T cells expansion in agitated conditions in spinner flasks was found to be higher, compared to static 2D culture (FIGS. 2A and 2B). Costariol et al., has shown that the expansion of primary T cells is increased with agitation speed in an automated STR. In agreement, an increase in T cell expansion yield at higher agitation rates in a STR (FIG. 2C) was observed. Cell growth accompanied by consumption of nutrients results in accumulation of lactate in the cell culture, which hinders efficient cell growth and quality of final cell therapy product. Continuous media perfusion enables fresh supply of nutrients and removal of harmful metabolites, while retaining the cells. However, owing to 5-10 µm diameter of T cells, cell escape and filter fouling have been major issues related to T cell culture media perfusion. Tangential flow filtration system provides an attractive solution as a cell retention system, where the movement of fluid enables media perfusion without fouling. Furthermore, ATF provides an additional advantage of self-cleaning induced by back flush of alternating flow. As the pore size of the hollow fibers in Repligen’s ATF is 0.2 µm, ATF was employed as a cell retention device for continuous media perfusion in STR. Initial testing of ATF for cell retention and media exchange resulted in successful perfusion of 1 VVD without filter fouling and cell loss (Table 1). Post inoculation of the STR with T cells at 0.5 ×10⁶ cells/mL, T cells were activated for 3 days with CD3+28 in the presence of recombinant human IL-2. After 3 days of activation, the cells were expanded in X-VIVO™ 15 media containing recombinant human IL-2. Monitoring lactate levels, a 3-fold increase in lactate levels from day 7 to day 8 was observed (FIG. 3C). This is accompanied by an increase in viable cell density to 2.1 ×10⁶ cells/mL (FIG. 3B) and a drop in glucose concentration to 5.4 mM on day 8 from 6.3 mM on day 7 (FIG. 3D), warranting media perfusion. Enabling media perfusion on day 8 has resulted in steady state levels of glucose and lactate (FIGS. 3C and 3D). Furthermore, continuous media perfusion has enabled exponential growth of T cells, yielding ~35 × 10⁶ cells/mL as final VCD. In comparison, T cells from same donor were activated and expanded in 1L G-Rex®. T cell expansion in G-Rex® resulted in a VCD of 3.4 × 10⁶ cells/mL on day 14, yielding a 28-fold expansion of T cells. A 14-day culture in STR with ATF mediated perfusion resulted in a 167-fold expansion of T cells (FIG. 3B). In addition, it was observed that agitation in STR in combination with ATF mediated cell movement did not decrease cell viability or cell expansion (FIG. 3E).

Turtle et al. reported in their clinical trials that CAR T cell therapy with a 1:1 CD4:CD8 T cell ratio resulted in 93% of the patients achieving bone marrow remissions [18]. FIG. 4A shows the maintenance of CD4:CD8 T cell ratio in the STR over 14 days expansion, suggesting a 1:1 seeding ratio would be maintained by harvest. Growing evidence suggests that the presence of high number of naive, stem cell memory and central memory T cells in the final CAR T cell therapy product resulted in relapse free remissions, compared to lower efficacy observed in CAR T cell product containing higher percentage of effector memory subset. T cell expansion in the tested conditions is shown to result in the final T cell subtype composition containing ~80% central memory subset, ~15% naive and stem cell memory subset, <10% effector memory and terminal differentiated subsets (FIG. 4B). This phenotype can be explained by higher replicative potential of naive T cells and stem cell memory T cells, which replicate and differentiate upon activation. The higher differentiation from naive and T memory stem cells (Tscm) combined with higher replicative potential of central memory T cells could have yielded higher percentage of central memory T cells. Even though final T cell subtype concentrations depend on the initial T cell composition, T cell expansion in Quantum yielded in similar phenotypic results. Presence of senescent and exhausted T cells in the final drug product results in lower efficacy explained by lower replication potential and dysfunctional state of the T cells, respectively. T cell expansion in STR was shown to result in >5% senescent (CD57+ KLRG1+) or exhausted T cells (CTLA4+/PD-1+) (FIG. 4C). Polyfunctional strength index™ (PSI) of T cells was predictive of clinical responses in Acute Myeloid Leukemia and could be used as biomarker in immunotherapy. PSI of the immune cells is calculated by multiplying the number of cytokines produced by each cell with the amount of each cytokine. Evaluation of PSI of CD4 + and CD8 + T cells after stimulation for 5 hours suggested the increase of effector CD4 + and CD8 + T cells from day 0 to 14. In addition to an increase in the effector signature, an increase in stimulatory signature, specifically in CD8 + T cells (FIG. 5D) was observed. Polyfunctional T cells are capable of producing 2+ cytokines upon stimulation with antigen, and is considered as a vital functional characteristic. Evaluation of the share of cells producing multiple cytokines suggested an increase in the percentage of CD4 + and CD8 + T cells producing multiple cytokines, specifically 5+ cytokines (FIG. 5B). Furthermore, ~1% CD4 + T cells have produced 11 cytokines and ~1% CD8 + T cells have produced 9 cytokines upon activation (FIG. 5C). As assessed by the phenotypic characterization of T cells, increase in the central memory phenotype could be translated into the increase in number of cells producing multiple cytokines.

Generation of allogeneic CAR T cells requires deletion of TCR alpha coding TRAC locus. Different strategies are employed to excise TRAC locus and they widely differ in their efficiency. CRISPR/Cas9 shows efficiency of 70-80%, TALEN - 60 to 80%, Zinc finger nucleases - 20 to 40% and megaTALs show an efficiency of 75%. Presence of TCR positive T cells in the allogeneic cell therapy product results in GVHD and have to be depleted before concentration and formulation. TCR positive T cell depletion requires harvesting T cells from STR and process through an exclusive unit, which increases the chance of contamination and incubation of cells in unoptimized conditions. Proprietary magnetic technology which facilitates cell depletion in STR, while maintaining optimized conditions was developed. In addition, cell depletion in STR avoids the need for an additional unit operation and decreases the chance of contamination. As a proof of principle, CD4+ T cells were depleted after T cell expansion. As shown in FIG. 6A and Table 2, 30 minutes of magnetic cell depletion effectively depletes CD4+ T cells at lower percentages (>99%) and higher percentages (97%). FDA guidelines requires the residual bead percentage of less than 0.003% in the final cell therapy product, ensuring minimal effect of beads in the system. Using magnetic depletion for 120 mins a decrease in the residual bead percentage to 0.001% without a cell loss and a significant drop in viability is shown (FIG. 6B and Table 3). Furthermore, the proprietary magnetic technology could potentially be scaled to yield similar results different scales.

Post depletion of undesired T cells, concentration and formulation is performed to decrease to the volume amicable for therapeutic use. Two automated, mutually exclusive and closed systems for cell processing and concentration were evaluated. ekko™ performs the concentration using acoustic technology and kSep concentrates the cells by fluidized bed formation using centrifugal force. As shown in Table 4, the cells were concentrated at least 7 fold, recovering >70% cells without a significant drop in cell viability.

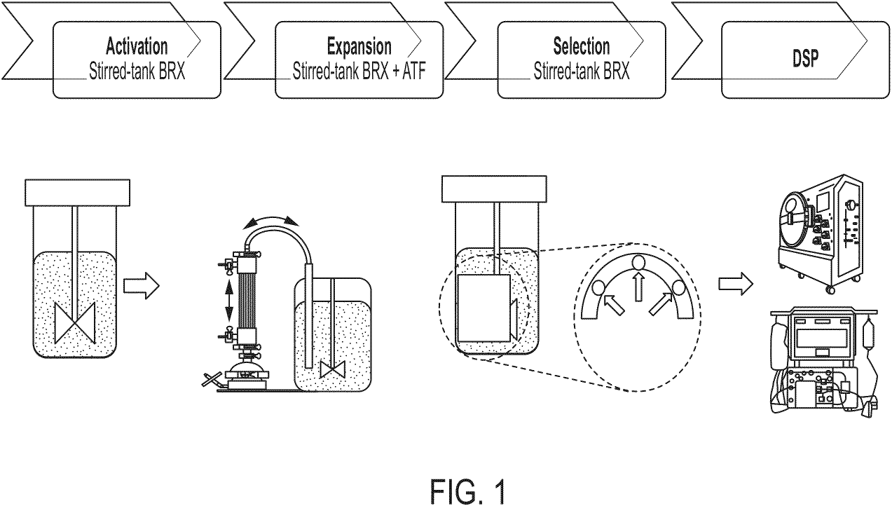

To date, this is the first study to describe the successful application of ATF for cell retention and media perfusion in T cell manufacturing. Collectively, this data demonstrates the capabilities of the closed, GMP compatible, end-to-end platform in expanding T cells to clinically relevant doses, in purifying cell cultures, in concentration and formulation of cell product (FIG. 1).

### ADDITIONAL EXEMPLARY EMBODIMENTS

Embodiment 1 is a method of producing an immune cell culture in a fully closed system, comprising obtaining an immune cell, introducing the immune cell into a stirred-tank bioreactor comprising an immune cell complete medium, activating the immune cell with an activation reagent to produce an activated immune cell in the stirred-tank bioreactor, expanding the activated immune cell to produce an expanded immune cell culture in the stirred-tank bioreactor, exchanging a defined amount of fresh medium for spent medium via an alternating tangential flow filtration (ATF) connected to the bioreactor, depleting the expanded immune cell culture to produce a depleted immune cell culture in the stirred-tank bioreactor, harvesting the depleted immune cell culture of to produce a harvested immune cell culture in the fully closed system and concentrating the harvested immune cell culture of (g) in the fully closed system, wherein the method results in less than 1% loss of the immune cell culture.

Embodiment 2 includes the method of embodiment 1, wherein the immune cell complete medium comprises a buffer, amino acids, trace elements, vitamins, inorganic salts, glucose and serum.

Embodiment 3 includes the method of embodiment 1, wherein the stirred-tank bioreactor has a volume capacity of about 1 L to about 2000 L.

Embodiment 4 includes the method of embodiment 1, wherein the immune cell is isolated from a population of peripheral blood mononuclear cells (PBMCs) immediately prior to obtaining the immune cell or is isolated from a population of PBMCs, stored for an extended period of time, and then thawed prior to obtaining the immune cell.

Embodiment 5 includes the method of embodiment 1, wherein the immune cell is derived from a population of pluripotent stem cells.

Embodiment 6 includes the method of embodiment 5, wherein the population of pluripotent stem cells is a population of induced pluripotent stem cells (iPSCs), embryonic stem cells (ESCs), or combinations thereof.

Embodiment 7 includes the method of embodiment 4, wherein isolating the immune cell comprises washing the PBMCs with a solution comprising antibody complexes and magnetic particles to produce a solution comprising the PBMCs and isolated T-cells and separating the isolated T-cells from the solution with a magnet

Embodiment 8 includes the method of embodiment 1, wherein between 0.25 ×10⁶ T-cells/mL and 2 × 10⁶ T-cells/mL are introduced into the bioreactor.

Embodiment 9 includes the method of embodiment 1, wherein the method produces an immune cell culture comprising about 10 × 10⁶ cells/mL to about 90 × 10⁶ cells/mL viable immune cells.

Embodiment 10 includes the method of embodiment 1, wherein the method produces an immune cell culture comprising at least about 100 million viable immune cells.

Embodiment 11 includes the method of embodiment 1, wherein the activation reagent comprises soluble antibody complexes.

Embodiment 12 includes the method of embodiment 1, wherein activating the immune cell comprises stirring the medium with the activation reagent for a period of about 72 hours at 37° C.

Embodiment 13 includes the method of embodiment 12, wherein the immune cell culture is stirred at a tip speed of 0.15 to 0.5 revolutions per minute (RPM).

Embodiment 14 includes the method of embodiment 1, wherein the medium has a pH between about pH 5.0 and about pH 7.5 during the activation period.

Embodiment 15 includes the method of embodiment 1, wherein the expanding comprises adding fresh medium to the bioreactor after the activation period, monitoring one or more of a temperature sensor, a pH sensor, a glucose sensor, an oxygen sensor, a carbon dioxide sensor, and an optical density sensor of the immune cell culture and adjusting one or more of a temperature, a pH level, a glucose level, an oxygen level, a carbon dioxide level, and an optical density of the immune cell culture, based on the monitoring.

Embodiment 16 includes the method of embodiment 15, wherein the expanding further comprises sampling the expanding immune cell culture, determining a cell growth and fold expansion of the expanding T cell culture and exchanging a defined amount of fresh medium for spent medium based on the cell growth and fold expansion.

Embodiment 17 includes the method of embodiment 16, wherein the fresh medium is exchanged for spent medium at a rate of one vessel volume per day (VVD) when the viable cell density of the expanding immune cell culture is greater than 1.5×106 cells/mL

Embodiment 18 includes the method of any of embodiment 1, wherein the depleting comprises adding surface-activated magnetic beads to the immune cell culture after the expanding, stirring the expanded immune cell culture and beads for about 30 minutes and isolating a population of target cells from the expanded immune cell culture with a magnet.

Embodiment 19 includes the method of embodiment 18, wherein the beads are added to the bioreactor at a bead-to-cell ratio of 1:1.

Embodiment 20 includes the method of embodiment 1, wherein the harvesting comprises pumping the immune cell culture from the stirred-thank bioreactor into a sterile container connected to the stirred-tank bioreactor and pumping the immune cell culture from the ATF into the sterile container connected to a harvest line of the ATF.

Embodiment 21 includes the method of embodiment 20, wherein the connection between the stirred-tank bioreactor and the ATF is closed.

Embodiment 22 includes the method of embodiment 20, wherein the pumping of the cell culture from the stirred-tank bioreactor into the sterile container is performed while the immune cell culture is continuously stirred in the stirred-tank bioreactor.

Embodiment 23 includes the method of embodiment 1, wherein the harvesting occurs after about 14 days of total cell culture.

Embodiment 24 includes the method of embodiment 1, wherein the concentrating comprises centrifugation, supernatant removal following sedimentation, filtration, acoustic cell processing, or combinations thereof of the harvested cell culture.

Embodiment 25 includes the method of embodiment 24, wherein the harvested cell culture is centrifuged at about 250 G to 3,000 G for about 60 minutes.

Embodiment 26 includes the method of embodiment 25, wherein centrifugation of the harvested cell culture produces a fluidized bed comprising a biomass of desired cells at a flow rate of about 20 mL/min to about 30 mL/min.

Embodiment 27 includes the method of embodiment 26, wherein the fluidized bed is pumped into a sterile and enclosed container for harvesting and/or storage.

Embodiment 28 includes the method of embodiment 24, wherein the acoustic cell processing comprises circulating the harvested cell culture through an acoustic fluid bed at 120 watts and a flow rate of about 50 mL/min to about 80 mL/min.

Embodiment 29 includes the method of embodiment 1, wherein the activating and the expanding of the immune cell in the stirred-tank bioreactor results in a T-cell culture producing greater than five cytokines, and with greater than 75% central memory T-cells, less than 10% effector memory T-cells, and greater than 10% naïve/stem memory T-cells.

Embodiment 30 includes the method of any of embodiment 1, wherein the concentrated T-cells are cryopreserved.

Embodiment 31 is a population of T-cells produced by a method according to embodiment 1.

Embodiment 32 is a T-cell based therapy comprising a T-cell produced by the method of embodiment 1.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method of producing an immune cell culture in a fully closed system, comprising:
a) obtaining an immune cell;
b) introducing the immune cell into a stirred-tank bioreactor comprising an immune cell complete medium;
c) activating the immune cell with an activation reagent to produce an activated immune cell in the stirred-tank bioreactor;
d) expanding the activated immune cell to produce an expanded immune cell culture in the stirred-tank bioreactor;
e) exchanging a defined amount of fresh medium for spent medium via an alternating tangential flow filtration (ATF) connected to the bioreactor;
f) depleting the expanded immune cell culture of (d) to produce a depleted immune cell culture in the stirred-tank bioreactor;
g) harvesting the depleted immune cell culture of (f) to produce a harvested immune cell culture in the fully closed system; and
h) concentrating the harvested immune cell culture of (g) in the fully closed system, wherein the method results in less than 1% loss of the immune cell culture.

2. The method of claim 1, wherein the immune cell complete medium comprises a buffer, amino acids, trace elements, vitamins, inorganic salts, glucose and serum.

3. (canceled)

4. The method of claim 1, wherein the immune cell is isolated from a population of peripheral blood mononuclear cells (PBMCs) immediately prior to obtaining the immune cell in a) or is isolated from a population of PBMCs, stored for an extended period of time, and then thawed prior to obtaining the immune cell in a).

5. The method of claim 1, wherein the immune cell is derived from a population of pluripotent stem cells.

6. (canceled)

7. The method of claim 4, wherein isolating the immune cell comprises a) washing the PBMCs with a solution comprising antibody complexes and magnetic particles to produce a solution comprising the PBMCs and isolated T-cells and b) separating the isolated T-cells from the solution with a magnet.

8. (canceled)

9. The method of claim 1, wherein the method produces an immune cell culture comprising about 10 x 10⁶ cells/mL to about 90 x 10⁶ cells/mL viable immune cells.

10. (canceled)

11. (canceled)

12. The method of claim 1, wherein activating the immune cell comprises stirring the medium with the activation reagent for a period of about 72 hours at 37° C.

13. The method of claim 12, wherein the immune cell culture is stirred at a tip speed of 0.15 to 0.5 revolutions per minute (RPM).

14. The method of claim 1, wherein the medium has a pH between about pH 5.0 and about pH 7.5 during the activation period.

15. The method of claim 1, wherein the expanding comprises:
i) adding fresh medium to the bioreactor after the activation period in (c);
ii) monitoring one or more of a temperature sensor, a pH sensor, a glucose sensor, an oxygen sensor, a carbon dioxide sensor, and an optical density sensor of the immune cell culture; and
iii) adjusting one or more of a temperature, a pH level, a glucose level, an oxygen level, a carbon dioxide level, and an optical density of the immune cell culture, based on the monitoring.

16. The method of claim 15, wherein the expanding further comprises iv) sampling the expanding immune cell culture, v) determining a cell growth and fold expansion of the expanding T cell culture and vi) exchanging a defined amount of fresh medium for spent medium based on the cell growth and fold expansion.

17. The method of claim 16, wherein the fresh medium is exchanged for spent medium at a rate of one vessel volume per day (VVD) when the viable cell density of the expanding immune cell culture is greater than 1.5x10⁶ cells/mL.

18. The method of claim 1, wherein the depleting comprises:
i) adding surface-activated magnetic beads to the immune cell culture after the expanding in d);
ii) stirring the expanded immune cell culture and beads for about 30 minutes; and
iii) isolating a population of target cells from the expanded immune cell culture with a magnet.

19. (canceled)

20. The method of claim 1, wherein the harvesting comprises i) pumping the immune cell culture from the stirred-thank bioreactor into a sterile container connected to the stirred-tank bioreactor and ii) pumping the immune cell culture from the ATF into the sterile container connected to a harvest line of the ATF.

21. (canceled)

22. (canceled)

23. The method of claim 1, wherein the harvesting occurs after about 14 days of total cell culture.

24. The method of claim 1, wherein the concentrating comprises centrifugation, supernatant removal following sedimentation, filtration, acoustic cell processing, or combinations thereof of the harvested cell culture.

25. (canceled)

26. The method of claim 25, wherein centrifugation of the harvested cell culture produces a fluidized bed comprising a biomass of desired cells at a flow rate of about 20 mL/min to about 30 mL/min.

27. The method of claim 26, wherein the fluidized bed is pumped into a sterile and enclosed container for harvesting and/or storage.

28. The method of claim 24, wherein the acoustic cell processing comprises circulating the harvested cell culture through an acoustic fluid bed at 120 watts and a flow rate of about 50 mL/min to about 80 mL/min.

29. The method of claim 1, wherein the activating and the expanding of the immune cell in the stirred-tank bioreactor results in a T-cell culture producing greater than five cytokines, and with greater than 75% central memory T-cells, less than 10% effector memory T-cells, and greater than 10% naïve/stem memory T-cells.

30. (canceled)

31. (canceled)

32. (canceled)
